(19) United States
(12) Patent Application Publication
(10) Pub. No.: US 2024/0052286 A1
(43) Pub. Date: Feb. 15, 2024

(54) SYSTEM AND METHOD OF EVALUATING CELL CULTURE

(71) Applicant: EMMAUS MEDICAL, INC., Torrance, CA (US)

(72) Inventors: Joan OLIVA VILANA, Lakewood, CA (US); Jun OCHIAI, Kettering, OH (US)

(21) Appl. No.: 18/260,415

(22) PCT Filed: Jan. 5, 2022
(86) PCT No.: PCT/US2022/011267

§ 371 Date: Jul. 5, 2023

Related U.S. Application Data
(60) Provisional application No. 63/205,757, filed on Jan. 6, 2021.

(51) Int. Cl. C12M 1/34; C12M 1/00

(52) U.S. Cl. CPC C12M 41/36; C12M 41/06

(57) ABSTRACT

Described herein is an apparatus for evaluating biological cell cultures. The apparatus comprises a housing and a controller coupled to the housing over a data bus. The housing comprises a light source to generate light, a collimator to collimate the light generated by the light source, a linear stage to actuate a cell culture dish including a biological cell culture in orthogonal directions, and a photodetector to receive the collimated light through the cell culture dish and the biological cell culture. The controller is configured to provide instructions to operate the light source, the linear stage, and the photodetector over the data bus.

### CROSS REFERENCE TO RELATED APPLICATIONS

This application claims priority under 35 U.S.C. § 119(e) to U.S. Provisional Application No. 63/205,757, filed Jan. 6, 2021, which is hereby incorporated by reference in its entirety.

### TECHNICAL FIELD

The present invention generally relates to evaluating biological substances. More particularly, the present inventions relate to a system and a method of determining thickness, maturity, and transparency of biological cell cultures.

### BACKGROUND

Cell sheet technology has gained interest in regenerative medicine to heal damaged organs or tissues. Cell sheet can be monolayer or multilayers. In regenerative medicine, sheets of laboratory grown stem cells can be transplanted onto damaged organs or tissues such that the transplanted stem cells can regenerate as cells of the underlying organs or tissues. Such techniques have been used successfully in skin grafting, for example. As another example, sheets transparent cell cultures, such as cornea cells, can be grown and transplanted onto damage eyes. In addition, cornea transmittance of donors can be measured with precision before transplantation. Under conventional methodologies, to grow such stem or cornea cell sheets, various scaffoldings such as amniotic membrane, fibrin gel, hyaluronan hydrogel, collagen, etc. are used to grow a thin stem or cornea cell sheet. The stem or cornea cell sheets are then harvested and stacked on top of other stem or cornea cell sheets to form a multilayered stem or cornea cell sheet which can then be transplanted onto a damaged organ or tissue for regeneration. These conventional methodologies can have many drawbacks. For example, a newly grown stem or cornea cell sheet needs to be mechanically removed or separated from a cell culture dish and then be placed on top of another stem or cornea cell sheet. In order for the stem or cornea cell sheet to withstand the force of being removed, the stem or cornea cell sheet must be grown long enough such that the stem or cornea cell sheet possesses strong enough physical integrity to retain sheet structure when lifted from the cell culture dish. If a stem or cornea cell sheet is prematurely harvested, the stem or cornea cell may tear and the process of growing a replacement stem or cornea cell sheet needs to be repeated. As such, the process of growing multilayered stem or cornea cell sheets can be laborious, time consuming, complex, and costly.

### SUMMARY

Described herein is an apparatus for determining thickness, maturity, transparency of biological cell cultures and the number of cells in the cell sheet. The apparatus can comprise a housing and a controller coupled to the housing over a data bus. The housing can comprise a light source to generate light, a collimator to collimate the light generated by the light source, a linear stage to actuate a cell culture dish including a biological cell culture in orthogonal directions, and a photodetector to receive the collimated light through the cell culture dish and the biological cell culture. The light source can be disposed at top of the housing. The collimator can be disposed below the light source. The linear stage can be disposed below the collimator and can provide a surface to secure the cell culture dish. The photodetector can be disposed below the linear stage and at a base on the housing. The controller can be configured to provide instructions to operate the light source, the linear stage, and the photodetector over the data bus.

In some embodiments, the housing can further comprise rods extending vertically from the base of the housing, a first bridge mechanically coupled to the rods, and a second bridge mechanically coupled to the rods. The first bridge can be disposed at the top of the housing and can include the light source. The second bridge can be disposed between the first bridge and the collimator and can include an aperture aligned to a line of sight of the light source.

In some embodiments, the collimator can comprise at least one lens. The lens can be mechanically coupled to a rod via an arm and an arm joint.

In some embodiments, the lens can have a lens diameter of 25.4 mm and a focal length of 25.4 mm.

In some embodiments, the lens can be housed in a bracket coupled to the arm.

In some embodiments, the linear stage can comprise a sample stage, an x-stage, and a y-stage. The sample stage can provide the surface to secure the cell culture dish. The x-stage can actuate the linear stage in a first direction. The y-stage can actuate the linear stage in a second direction orthogonal to the first direction.

In some embodiments, the x-stage and the y-stage can include a stepper motor coupled to a timing belt that causes the x-stage and the y-stage to move in their respective directions.

In some embodiments, each of the sample stage, the x-stage, and the y-stage can include an opening that allows the collimated light to pass through.

In some embodiments, the controller can comprise a computing unit coupled to at least two motor drive modules.

In some embodiments, the at least two motor drive modules can generate signals to actuate the stepper motors of the x-stage and the y-stage.

In some embodiments, the computing unit can receive analog signals from the photodetector over the data bus and digitize the analog signals.

In some embodiments, the computing unit can generate digital signals to turn the photodetector on or off.

In some embodiments, the light generated by the light source can comprise a sharp peak at 450-475 nm and a flatten peak at 560-60 nm.

In some embodiments, the data bus can be a wired data connection.

In some embodiments, the wired data connection can be at least one of an ethernet, serial, or general purpose interface bus based data connection.

In some embodiments, the data bus can be a wireless data connection.

In some embodiments, the wireless data connection can be at least one of a cellular, Wi-Fi, Bluetooth, or near-field communication based data connection.

Described herein is a method for operating the apparatus. The controller can actuate the linear stage to a first location corresponding to a first predetermined location of the cell culture dish. The light source through the collimator can generate collimated light to pass through the cell culture dish and the biological cell culture at the first location. The photodetector can receive the collimated light passing through the cell culture dish and the biological cell culture. The controller can determine intensity of the collimated light at the first location.

In some embodiments, the intensity of the collimated light can be an average of at least 10 intensity measurements.

In some embodiments, the controller can actuate the linear stage to a second location corresponding to a second predetermined location of the cell culture dish. The light source through the collimator can generate collimated light to pass through the cell culture dish and the biological cell culture at the second location. The photodetector can receive the collimated light passing through the cell culture dish and the biological cell culture. The controller can determine intensity of the collimated light at the second location.

These and other features of the apparatuses, systems, methods, and non-transitory computer-readable media disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for purposes of illustration and description only and are not intended as a definition of the limits of the invention.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the invention are utilized, and the accompanying drawings of which:

FIGS. 1A-1D illustrate an apparatus for determining thickness, maturity, and transparency of a biological cell culture, according to various embodiments of the present disclosure.

FIG. 2 illustrates an electrical schematic of the controller, according to various embodiments of the present disclosure.

FIG. 3A illustrates a visual representation of a configuration setting can be loaded onto the controller to measure light intensity, according to various embodiments of the present disclosure.

FIG. 3B illustrates a method to operate the apparatus, according to various embodiments of the present disclosure.

FIG. 3C illustrates a method to determine transmittance of biological cell cultures using the apparatus, according to various embodiments of the present disclosure.

FIG. 4A illustrates a diagram showing a relationship between transmittance (light intensity) and thickness, or the harvesting time (named also maturity) of a biological cell culture, according to various embodiments of the present disclosure.

FIGS. 4B-4C illustrate graphs depicting a relationship between transmittance of a biological cell culture, such as a multilayered stem or cornea cell sheet, and days the biological cell culture was grown in a laboratory environment, according to various embodiments of the present disclosure.

FIG. 4D illustrates a method of determining thickness and/or maturity of a biological cell culture, such as a multilayered stem or cornea cell sheet, according to various embodiments of the present disclosure.

FIG. 4E illustrates a reference graph that can be used to determine a number of cells in a cell sheet, according to various embodiments of the present disclosure.

The figures depict various embodiments of the disclosed technology for purposes of illustration only, wherein the figures use like reference numerals to identify like elements. One skilled in the art will readily recognize from the following discussion that alternative embodiments of the structures and methods illustrated in the figures can be employed without departing from the principles of the disclosed technology described herein.

### DETAILED DESCRIPTION

In regenerative medicine, sheets of laboratory grown stem cells can be transplanted onto damaged organs or tissues such that the transplanted stem cells can regenerate as cells of the underlying organs or tissues. Such techniques have been used successfully in skin grafting, for example. As another example, sheets transparent cell cultures, such as cornea cells, can be grown and transplanted onto damage eyes, or cornea transmittance of donors can be measured with precision before transplantation. Under conventional methodologies, to grow such stem or cornea cell sheets, various scaffoldings such as amniotic membrane, fibrin gel, hyaluronan hydrogel, collagen, etc. are used to grow a thin stem or cornea cell sheet. The stem or cornea cell sheets are then harvested and stacked on top of other stem cell sheets to form a multilayered stem or cornea cell sheet which can then be transplanted onto a damaged organ or tissue for regeneration. These conventional methodologies can have many drawbacks. For example, a newly grown stem or cornea cell sheet needs to be mechanically removed or separated from a cell culture dish and then be placed on top of another stem or cornea cell sheet. In order for the stem or cornea cell sheet to withstand the force of being removed, the stem or cornea cell sheet must be grown long enough such that the stem or cornea cell sheet possesses strong enough physical integrity to retain sheet structure when lifted from the cell culture dish. If a stem or cornea cell sheet is prematurely harvested, the stem or cornea cell may tear and the process of growing a replacement stem or cornea cell sheet needs to be repeated. As such, the process of growing multilayered stem or cornea cell sheets can be laborious, time consuming, complex, and costly. Better solutions are needed for monitoring multilayered stem or cornea cell sheets growth, but also to determine the harvesting time of the cell sheets and the number of cells which is part of the cell sheets posology.

Described herein are inventions that address the problems described above. Unlike the conventional methodologies described above, the inventions described herein can be used to monitor the growth of a multilayered stem or cornea cell sheet directly on a cell culture dish and to determine the harvesting time of the multilayered stem or cornea cell sheet so that it can be directly transplanted onto damaged organs or tissues. This methodology will allow also to determine the number of cells per cell sheets, using a non-invasive approach, which will be used for the posology. In this way, instead of growing stem or cornea cell sheets one by one and layering the stem or cornea cell sheets, a multilayered stem or cornea cell sheets can be directly grown to a particular thickness and/or maturity, then harvested. In various embodiments, the inventions can include an apparatus for determining thickness, maturity, and transparency of a biological cell culture, such as a multilayered stem or cornea cell sheet. The apparatus can comprise a housing that includes a light source, a collimator, a linear stage, a light detector, and a controller. The light source can be configured to generate (or emit) light at one or more frequencies. For example, in some embodiments, the light source can be configured to generate light at frequencies corresponding to red, white, or blue light. The collimator can collect the light emitted from the light source and convert the light into collimated light (i.e., parallel light rays) that can be focused onto an opening (e.g., an aperture) of the linear stage. A cell culture dish including the biological cell culture can be secured onto the linear stage such the collimated light can pass through the biological cell culture through the opening. The light detector can be configured to receive (or detect) the collimated light passing through the biological cell culture to measure its intensity. The controller can be configured to control the light source to output light at various frequencies. The controller can be configured to actuate the linear stage. For example, in some embodiments, the controller can include a drive circuit that can generate to signals to actuate the linear stage in x- and y-directions. And the controller can be further configured to determine transmittance of the biological cell culture based on the intensity. The apparatus will be discussed in further detail herein.

In various embodiments, the inventions can further include a method of determining thickness, maturity, and transparency of a biological cell culture using the apparatus. A cell culture dish including the biological cell culture can be placed on the linear stage. Collimated light emitted from the light source can be illuminated onto a number of predetermined locations of the cell culture dish such that the collimated light can pass through the cell culture and the biological cell culture at the predetermined locations. Intensities of the collimated light at the predetermined locations can be measured to determine transmittance values of the biological cell culture at the predetermined locations. Based on these transmittance values, thickness and/or maturity of the biological cell culture can be determined. These and other features of the inventions are described in further detail herein.

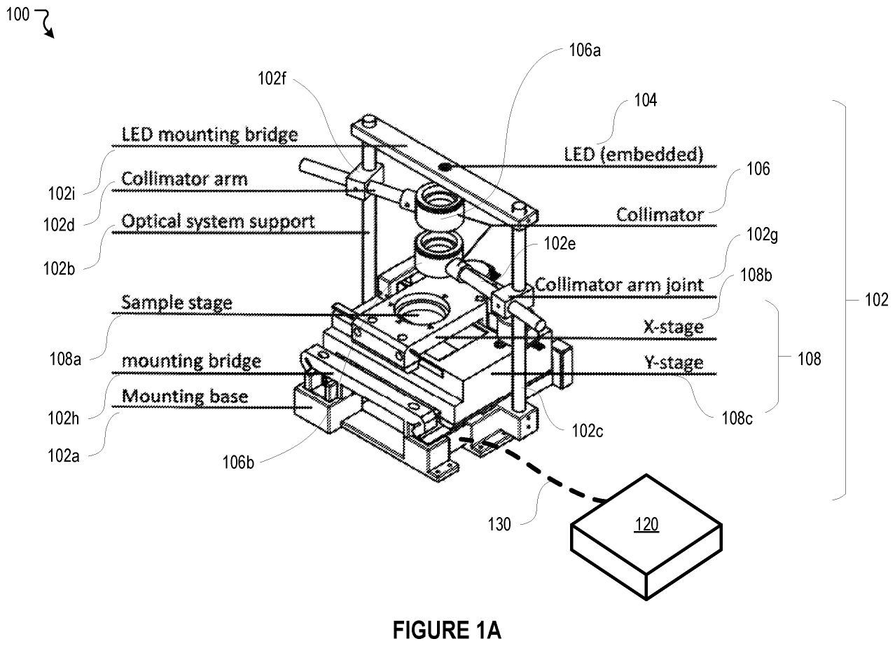

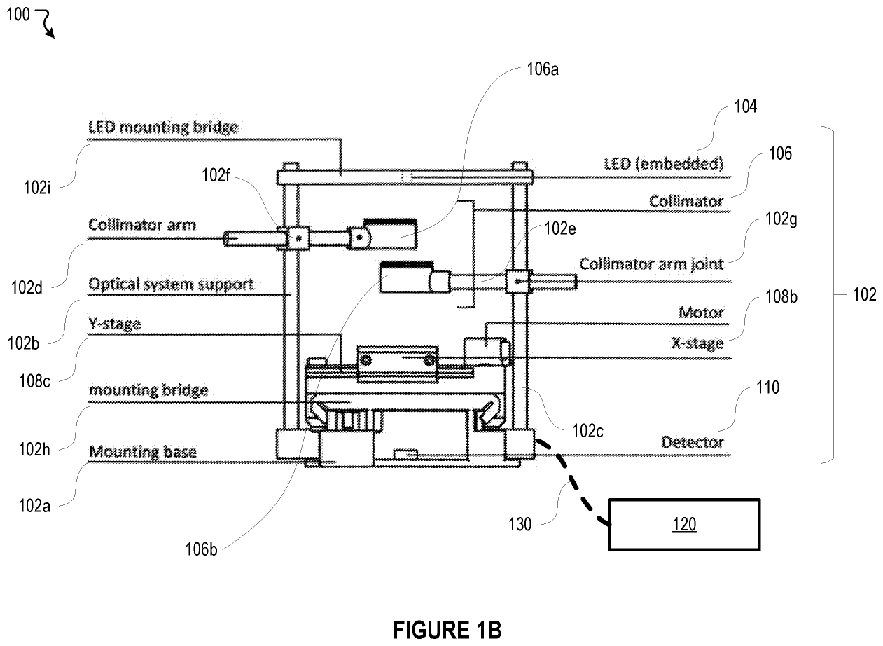

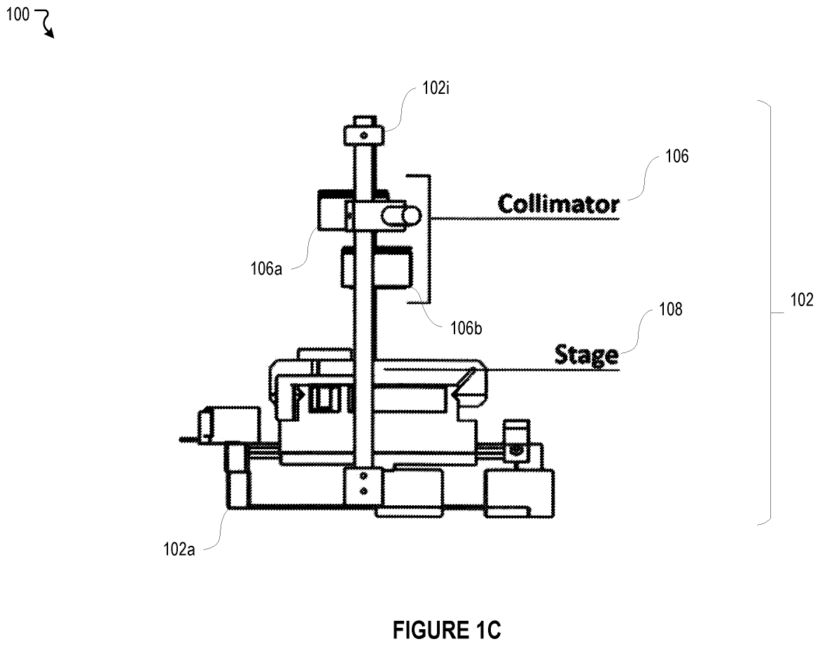

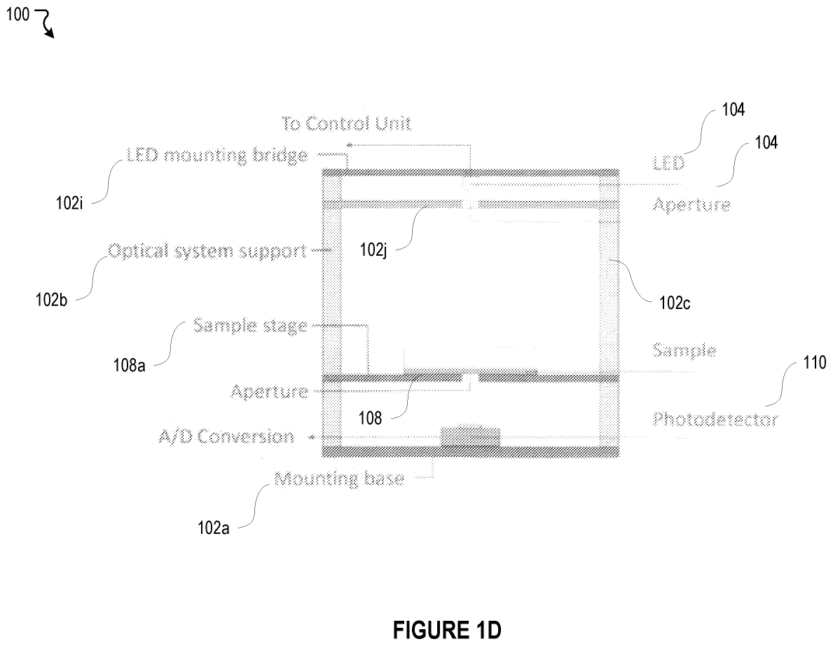

FIGURES IA-1D illustrate an apparatus 100 for determining thickness, maturity, and transparency of a biological cell culture, according to various embodiments of the present disclosure. FIG. 1A depicts an isometric view of the apparatus 100. FIG. 1B depicts a frontal view of the apparatus 100. FIG. 1C depicts a side view of the apparatus 100. FIG. 1D depicts a simplified frontal view of the apparatus 100. As shown in FIGS. 1A and 1B, in some embodiments, the apparatus 100 can comprise a housing 102 coupled to a controller 120 over a data bus 130. Although the housing 102 and the controller 120 are shown in FIGS. 1A and 1B as separate entities, in some embodiments, the controller 120 can be integrated into or be a part of the housing 102. In some embodiments, the data bus 130 can be a wired data connection. For example, the data bus 130 can be an ethernet, serial, or general purpose interface bus based data connection. In some embodiments, the data bus 130 can be a wireless data connection. For example, the data bus 130 can be a cellular, Wi-Fi, Bluetooth, or near-field communication data connection.

The housing 102 can include a light source 104, a collimator 106, a linear stage 108, and a photodetector 110 (shown in FIGS. 1B and 1D). In some embodiments, the housing 102 can further include a mounting base 102a that can be used to anchor the housing 102 onto a surface. For example, the housing 102 can be affixed on a fixture or a table through anchoring holes of the mounting base 102a in a laboratory environment. The mounting base 102a can include optical system support columns 102b, 102c that extend vertically from top of the mounting base 102a. Each of the optical system support columns 102b, 102c can include a collimator arm (i.e., 102d and 102e) that can be used to anchor one or more lenses of the collimator 106. For example, in some embodiments, the collimator 106 can comprise an optical system comprising at least two lenses that convert light into collimated light. In this example, a first lens of the optical system can be secured to the housing 102 through the collimator arm 102d and a second lens of the optical system can be secured to the housing 102 through the collimator arm 102e. In some embodiments, each of the collimator arms 102d, 102e can include a collimator arm joint (i.e., 102f and 102g) that can be used to mechanically couple the collimator arms 102d, 102e to the optical system support columns 102b, 102c, respectively. Each of the collimator arm joints 102f, 102g includes at least two openings that can be used to adjust height and length of the collimator arms 102d, 102e. For example, a first opening of the collimator arm joint 102f can be used to raise or lower height of the collimator arm 102d along the optical system support rod 102b. In some cases, the first opening can be used to rotationally move the lens attached to the collimator arm 102d in and out of a line of sight of light. A second opening of the collimator arm joint 102f can be used to lengthen or shorten the collimator arm 102d with respect to the optical system support rod 102b. Once the collimator arm joint 102f has been configured to be at the right height, rotation, and length, the collimator arm joint 102f can be secured to the optical system support rod 102b by tightening securing pins associated with the first opening and the second opening. The collimator arm joint 102g can be configured similarly to raise or lower height of the collimator arm 102e, or to lengthen or shorten the collimator arm 102e with respect to the optical system support rod 102c. In this way, the optical system of the collimator 106 can be adjusted in multitude of ways. For example, a focus of the collimator 106 can be adjusted by raising or lowering one or both of the collimator arms 102d, 102e. As another example, the collimator 106 can be moved in and out of the line of sight by lengthening or shortening one or both of the collimator arms 102d, 102e. Alternatively, the collimator 106 can be moved in and out of the line of sight by rotating one or both of the collimator arms 102d, 102e away from the line of sight.

In some embodiments, the housing 102 can further include at least one mounting bridge 102h that is mechanically coupled to the mounting base 102a. The mounting bridge 102h can provide structural rigidity to the mounting base 102a and, thus, the housing 102. For example, the mounting bridge 102 can minimize twisting or deformation to the mounting base 102a caused by actuation of the linear stage 108. In some cases, the mounting bridge 102h in conjunction with the mounting base 102 can provide a mounting spot at which to secure the linear stage 108. For example, in some embodiments, the mounting bridge 102h and the mounting base 102a can have through holes with which to secure the linear stage 108 to the housing 102.

In some embodiments, the housing 102 can further include a light source mounting bridge 102i coupled to the optical system support columns 102b, 102c. Similar to the mounting bridge 102h, the light source mounting bridge 102i can provide structural rigidity to the housing 102 when the linear stage 108 is being actuated. In some embodiments, the light source mounting bridge 102i can include a hole to embed (or integrate) the light source 104. Although the hole of the light source mounting bridge 102i is shown in FIGS. 1A-1C to be centrally located, in some embodiments, the hole can be disposed at other locations of the light source mounting bridge 102i. For example, the light source 104 can be embedded into the light source mounting bridge 102i off-center. Many variations are possible and contemplated.

In some embodiments, the housing 102 can further include an aperture bridge 102j (shown in FIG. 1D) coupled to the optical system support columns 102b, 102c. Similar to the mounting bridge 102h and the light source mounting bridge 102i, the aperture bridge 102j can provide structural rigidity to the housing 102 when the linear stage 108 is being actuated. In some embodiments, the aperture bridge 102j can further include an aperture (i.e., an opening) that allows light emitted from the light source 104 to pass and creating a line of sight. In general, the aperture bridge 102j and the light source mounting bridge 102i are disposed along the optical system support columns 102b, 102c such the aperture of the aperture bridge 102j is immediately below the light source 104 embedded into the light source mounting bridge 102i. In this way, stray light emitted from the light source is minimized before reaching the collimator 106. In one particular embodiment, the aperture bridge 102j is disposed along the optical system support columns 102b, 102c such that the aperture is 5 mm in distance from the light source 104.

Although the housing 102 as shown in FIGURES IA-1D is depicted as having an open structure, in some embodiments, the housing 102 can be fully enclosed in an enclosure. In such embodiments, interior of the enclosure can be lined with matte black foil so that ambient light cannot penetrate and interfere with the photodetector 110. For example, ambient light entering into the enclosure may increase light intensity seen by the photodetector 110. By lining the interior with the matte black foil, ambient light entering the enclosure can be reduced or eliminated entirely, thereby increasing accuracy of intensity determination. In some embodiments, the housing 102 can further include one or more cameras. The cameras can be further configured to control and focus light onto various locations on the linear stage 108. For example, the cameras can provide feedback to the linear stage 108 so that focusing of light onto various locations on the linear stage 108 can improve.

The light source 104 can be configured to generate light at one or more frequencies. In general, any type of light source can be implemented as the light source 104 and the light source 104 can be interchangeable. For example, in some embodiments, a halogen light source, a fluorescent light source, or an incandescent light source can be implemented as the light source 104. In other embodiments, an ultra-violet light source or an infra-red light source can be implemented as the light source 104. Many variations are possible and contemplated. In one particular embodiment, a light-emitting diode (LED) light source can be implemented as the light source 104. Implementing the light source 104 using LEDs can offer several advantages. For example, a LED light source can be configured or programmed to generate light at different frequencies or wavelengths. For instance, the LED light source can be instructed, via a control signal from the controller 120, for example, to output red, white, or blue light (i.e., at different wavelengths of light). Further, the LED light source can be adapted to have a small footprint so that it can be easily embedded into the light source mounting bridge 102i. In some embodiments, the light source 104 can be configured to emit phosphor-converted white light. The phosphor-converted white light can have a broad spectral power distribution with a sharp peak at 450-475 nm for blue color and a flatten peak at 560-60 nm for yellow light. Such light characteristics can facilitate illumination through a cell culture dish and a biological cell culture. In some embodiments, the light source 104 can be further adapted to generate particular wavelengths of light based on types of protein expressed by a biological cell culture. For example, if a biological cell culture expresses (or generates) green fluorescent protein, the light source 104 can be adapted to generate light that can detect presence of the green fluorescent protein, in addition to determining thickness and/or maturity of the biological cell culture. Many variations are possible and contemplated.

The collimator 106 can be configured to convert light into collimated light. The collimated light (i.e., parallel light rays) can be condensed (or focused) so that the collimated light rays can pass through an opening of the linear stage 108 and to the photodetector 110. As shown in FIGURES IA-1C, in some embodiments, the collimator 106 can comprise an optical system comprising at least two lenses 106a, 106b. The lenses 106a, 106b can be affixed to the collimator arms 102d, 102e, respectively. For example, the lenses 106a, 106b can be secured into brackets. The brackets can then be screwed onto the collimator arms 102d, 102e. In some embodiments, the lenses 106a, 106b can be plano-convex lenses to reduce stray light as light passes through the collimator 106. In general, the lenses 106a, 106b can have any lens diameters and focal lengths to collimate light. Selection of particular lens diameters for the lenses 106a, 106b can depend on various factors, such as lens materials and curvature of lenses, etc., for example. In one particular embodiment, at least one of the lenses 106a, 106b can have a lens diameter of 25.4 mm and a focal length of 25.4 mm. In other embodiments, both of the lenses 106a, 106b can have a lens diameter of 25.4 mm and a focal length of 25.4 mm.

The linear stage 108 can be configured to actuate a sample stage 108a in two orthogonal directions. For example, the linear stage 108 can be instructed, via one or more control signals generated by the controller 120, for example, to move the sample stage 108a along an x-axis or a y-axis of an x-y plane. In some embodiments, the linear stage 108 can further include an x-stage 108b coupled to a y-stage 108c. In some embodiments, the sample stage 108a can include an opening with a structural guide that allows a cell culture dish to be precisely placed onto the sample stage 108a. In this way, cell culture dishes including different biological cell cultures can be placed onto the sample stage 108a without affecting alignment of the cell culture dishes to the sample stage 108a. Each of the x-stage 108b and the y-stage 108c can include an opening that allows light to pass through. Each of the x-stage 108b and the y-stage 108c can further include a motorized, belt-driven actuator that allows the x-stage 108b and the y-stage 108c to be actuated in a linear direction. For example, the actuator of the x-stage 108b can move the x-stage 108b along the x-axis and the actuator of the y-stage 108c can move the y-stage 108b along the y-axis. In this way, a cell culture dish including a biological cell culture placed on the sample stage 108a can be moved to various locations so that collimated light exiting the collimator 106 can illuminate (or shine) through the cell culture dish and biological cell culture at those locations. In some embodiments, the motorized, belt-driven actuator can comprise a timing belt and a stepper motor. In such embodiments, the controller 120 can generate command signals to the stepper motor that cause the stepper motor to rotate to a particular rotational position. This rotation causes a stage that the stepper motor is coupled to, to actuate to a particular linear position through the timing belt. In some embodiments, each of the x-stage 108b and the y-stage 108c can include a lead screw-controlled actuator. Unlike the motorized, belt driven actuator, the lead screw controlled actuator improved precision and repeatability. In some embodiments, as shown in FIGURES IA-1C, the sample stage 108a can be integrated or be a part of the x-stage 108b. In other embodiments, the sample stage 108a can be mounted on top of the x-stage 108b.

The photodetector 110 can be configured to measure intensity of light passing through the cell culture dish and the biological cell culture secured onto the linear stage 108. In some embodiments, the photodetector 110 can be a monolithic photodiode. The photodetector 110 can convert intensity of light seen by the photodetector 110 into an analog voltage signal. This analog voltage signal can be digitized by an analog-to-digital converter of the controller 120. In general, the analog-to-digital converter can be of any suitable resolution. For example, in some embodiments, the analog-to-digital converter can have a resolution of 8-bits. In other embodiments, the analog-to-digital converter can have a resolution of 10-bits. Many variations are possible. In some embodiments, the photodetector 110 can be configured to continuously measure intensity of light. For example, in some embodiments, the photodetector 110 can be configured to measure intensity of light at 10 Hz (i.e., 10 intensity measurements per second). As another example, in some embodiments, the photodetector 110 can be configured to measure intensity of light at 100 Hz (i.e., 100 intensity measurements per second). In this way, intensity values that are of statical significance can be determined.

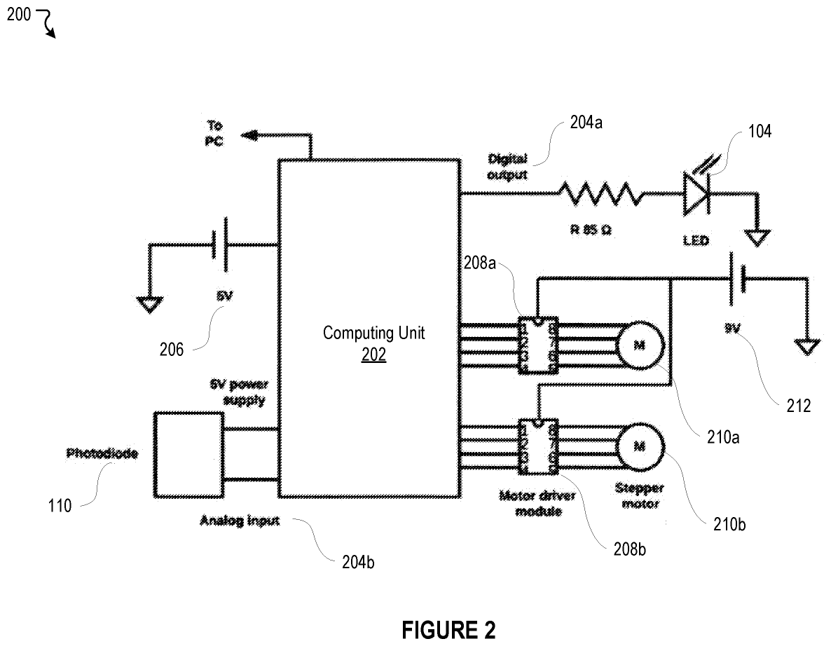

The controller 120 can be configured to control the light source 104, the linear stage 108, and the photodetector 110. For example, the controller 120 can transmit control signals, over the data bus 130, to the light source 104 to instruct the light source 104 to output light at particular frequencies or wavelengths. For instance, the controller 120 can instruct the light source 104 to output white light having a sharp peak at 450-475 nm for blue color and a flatten peak at 560-60 nm for yellow light. As another example, the controller 120 can transmit control signals, over the data bus 130, to the linear stage 108 to instruct the linear stage 108 to move to a particular position in an x-y plane. For instance, the controller 120 can instruct the linear stage 108 to move in positive x-direction by 10 mm and move in negative y-direction by 5 mm. As yet another example, the controller 120 can transmit control signals, over the data bus 130, to the photodetector 110 to instruct the photodetector 110 to measure light intensity at a particular rate. For instance, the controller 120 can instruct the photodetector 110 to measure light intensity at 5 Hz, 10 Hz, etc. In some embodiments, the controller 120 can synchronize operations associated with the light source 104, the linear stage 108, and the photodetector 110 such that light intensity measurements through the cell culture dish and the biological cell culture are automated. For example, in one particular implementation, the controller 120 can be configured to automatically measure light intensity at 9 predetermined locations of a cell culture dish including a biological cell culture and, at each location, measure light intensity at 10 Hz (i.e., 10 times). In this example, the controller 120 can synchronize operations of the light source 104, the linear stage 108, and the photodetector 110. For instance, the controller 120 first transmits control signals to the linear stage 108 to cause the linear stage 108 to move to a first predetermined location. Once the move is complete, the controller 120 transmits control signals to the light source 104 to output white light. Then finally, the controller 120 transmits control signals to the photodetector 110 to measure light intensity 10 times. Many variations are possible and contemplated. In some embodiments, the controller 120 can be implemented using a computing unit, such as an Arduino computing unit. The controller 120 will be discussed in further detail with reference to FIG. 2 herein.

FIG. 2 illustrates an electrical schematic 200 of the controller 120, according to various embodiments of the present disclosure. As shown in FIG. 2, in some embodiments, the controller 120 can include a computing unit 202. The computing unit 202 can include at least one processor, at least one memory, and at least one input/output interface coupled to each other over a data bus. In some embodiments, the computing unit 202 can further include one or more circuits to generate and/or read various signals to support operations of the light source 104, the linear stage 108, and the photodetector 110 through the input/output interface. For example, the computing unit 202 can include a circuit that generates a digital output signal 204a that causes the light source 104 to turn on or off. As another example, the computing unit 202 can further include a circuit that can receive an analog input signal 204b from the photodetector 110 and digitize the analog input signal 204b. In some embodiments, shown in FIG. 2, the computing unit 202 can be powered via an external 5V source 206. This 5V source 206 can power the processor, the memory, and the one or more circuits to generate and/or read various signals associated with the computing unit 202. In some embodiments, the controller 120 can further include at least two motor driver modules 208a, 208b. In such embodiments, the controller 120 can generate signals to the motor driver modules 208a, 208b causing the motor driver modules 208a, 208b to generate pulse width modulated signals to stepper motors 210a, 210b of the x-stage 108b and the y-stage 108c, respectively. The pulse width modulated signal can cause the stepper motors 210a, 210b to rotate, which in turn, causes the x-stage 108b and the y-stage 108c to linearly actuate. In some embodiments, the motor driver modules 208a, 208b can be powered by an external 9V source 212. In other embodiments, the motor driver modules 208a, 208b can be powered by the 5V source 206 powering the computing unit 202. In some embodiments, the computing unit 202 can be implemented using an Arduino computing unit, such as Arduino Uno R3. In other embodiments, the computing unit can be implemented using other suitable embedded computing systems.

In some embodiments, the computing unit 202 can be communicatively coupled to a computing system. The computing system can be configured to run an integrated development environment that enables a user (e.g., a programmer) to configure the computing unit 202 to perform automation. In general, the integrated development environment can support a plurality of programming languages for automation. For example, in one particular implementation, a user can program the computing unit 202 to perform automation using Python or Visual Basics code. Many programming languages are contemplated.

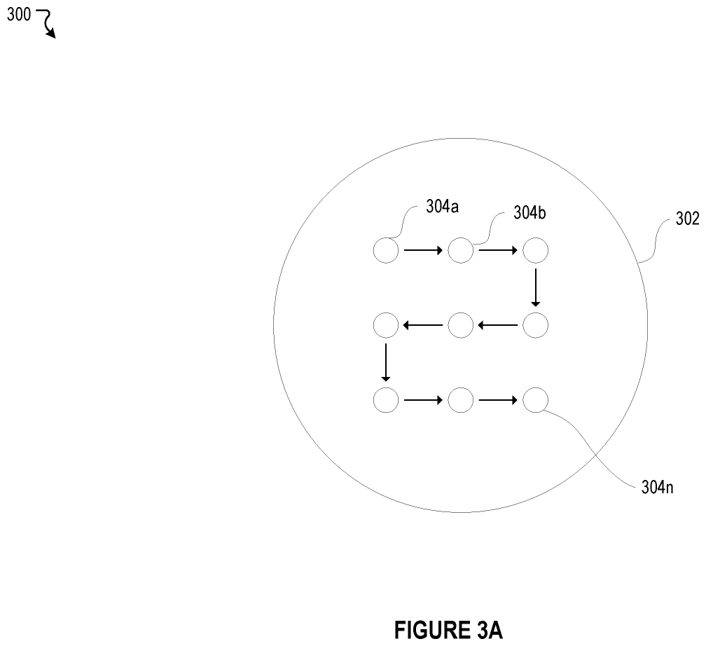

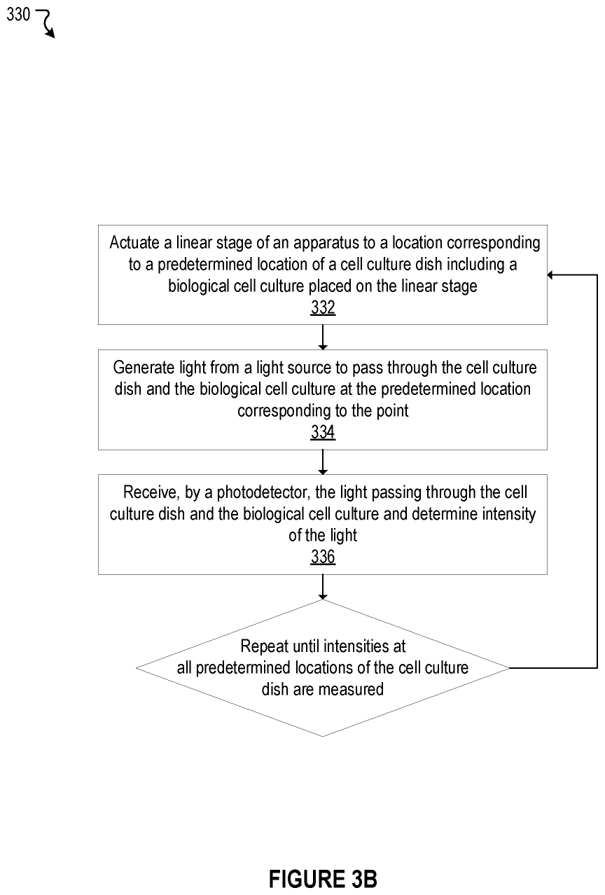

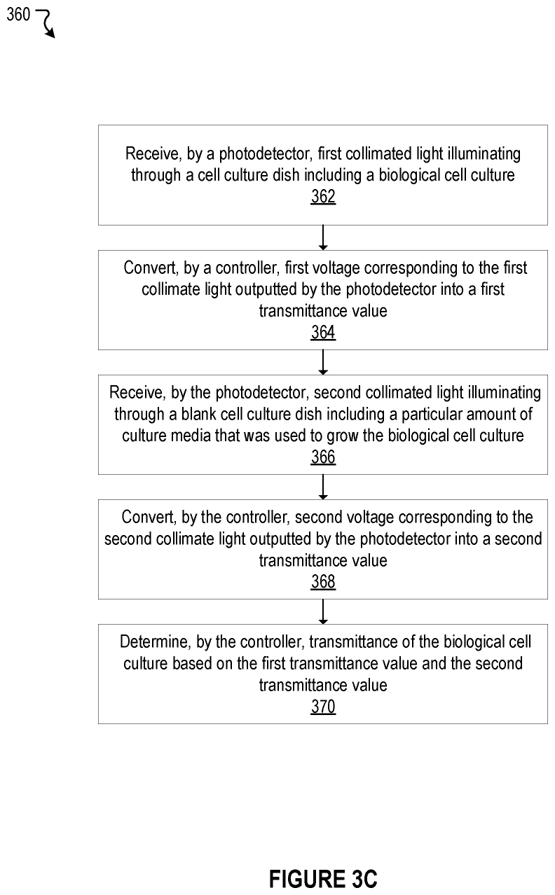

FIG. 3A illustrates a visual representation 300 of a configuration setting that can be loaded onto the controller 120 to measure light intensity, according to various embodiments of the present disclosure. As discussed above, in various embodiments, the controller 120 can be configured to automate light intensity measurements through a cell culture dish including a biological cell culture. This can be done, for example, by programming a configuration setting for the controller 120 through an integrated development environment running on a computing system coupled to the controller 120. The configuration setting can be loaded onto the controller 120 so that the controller 120 can synchronize operations of the light source 104, the linear stage 108, and the photodetector 110 for automation. As shown, FIG. 3A depicts a cell culture dish 302. The cell culture dish 302 can include a biological cell culture, such as a multilayered stem or cornea cell sheet, that was grown under laboratory conditions. The cell culture dish 302 can be secured onto the linear stage 108. The configuration setting can include instructions for the controller 120 to send to the light source 104, the linear stage 108, and the photodetector 110 in some particular order or sequence. For example, as a non-limiting example, the configuration setting can include a first set of instructions for the controller 120 to move the linear stage 108 to a location so that a line of sight of light emitted from the light source 104 intersects a first point 304a of the cell culture dish 302 and, thus, the biological cell culture. After some time delay to allow the linear stage 108 to move to the location, the configuration file can include a second set of instructions for the controller 120 to turn on the light source 104. After some more time delay to allow the light source 104 to turn on, the configuration file can include a third set of instructions for the controller 120 to instruct the photodetector to measure light intensities. Upon completion of light intensity measurements at the location corresponding to the first point 304a, the configuration file can include further instructions to cause the linear stage 108 to move to a location corresponding to a second point 304b of the cell culture dish 302 and repeat light intensity measurements at that location. These instructions continue until light intensity measurements are measured at all of the points 304a-304n of the cell culture dish 302. In some embodiments, the configuration setting can include instructions to move the cell culture dish in a serpentine-like route shown in FIG. 2.

FIG. 3B illustrates a method 330 to operate the apparatus 100, according to various embodiments of the present disclosure. At step 332, the linear stage 108 can be instructed to actuate to a location corresponding to a first predetermined location of a cell culture dish including a biological cell culture secured onto the linear stage 108. At step 334, the light source 104 can be instructed to generate light to pass through the cell culture dish and the biological cell culture at the location corresponding to the first predetermined location. At step 336, the photodetector 110 can receive the light passing through the cell culture dish and the biological cell culture and measure intensity of the light. This process is repeated until intensities at all predetermined locations of the cell culture dish are measured.

The techniques described herein, for example, are implemented by the controller 120. In some embodiments, the techniques described herein can be implemented by one or more special-purpose computing devices. The special-purpose computing devices may be hard-wired to perform the techniques, or may include circuitry or digital electronic devices such as one or more application-specific integrated circuits (ASICs) or field programmable gate arrays (FPGAs) that are persistently programmed to perform the techniques, or may include one or more hardware processors programmed to perform the techniques pursuant to program instructions in firmware, memory, other storage, or a combination.

FIG. 3C illustrates a method 360 to determine transmittance of biological cell cultures using the apparatus 100, according to various embodiments of the present disclosure. At step 362, collimated light illuminating through a cell culture dish including a biological cell culture is received by the photodetector 110. The photodetector 110 converts intensity of the collimated light into voltage. At step 364, an analog-to-digital converter of the controller 120 converts the voltage into a first digital value. The first digital value can range from 0 to 900, where 0 is equivalent to 0% transmittance (i.e., opaque) and 900 is equivalent to 100% transmittance (i.e., clear). At step 366, collimated light illuminating through a blank cell culture dish including 2 mL of culture media (e.g., osteoblast, chondrocyte, or undifferentiated culture media) that was used to grow the biological cell culture is received by the photodetector 110. At step 366, the analog-to-digital converter of the controller 120 converts voltage corresponding intensity of the collimated light illuminating through the blank cell culture dish into a second digital value. At step 368, transmittance of the biological cell culture is calculated based on the following expression:

(Cell Sheet Measurement/Blank Measurement)*100=% Transmittance

where cell sheet measurement is the transmittance of the cell culture dish including the biological cell culture and blank measurement is the transmittance of the blank cell culture dish including the culture media.

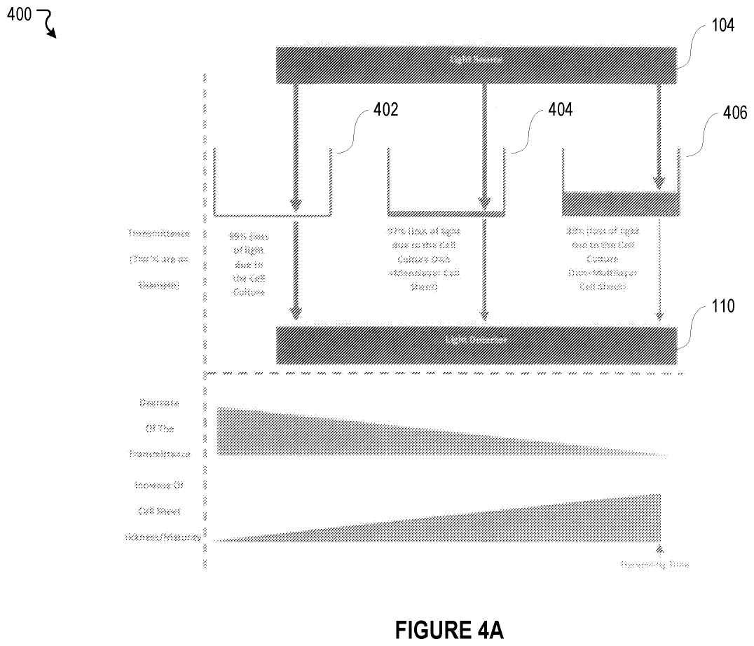

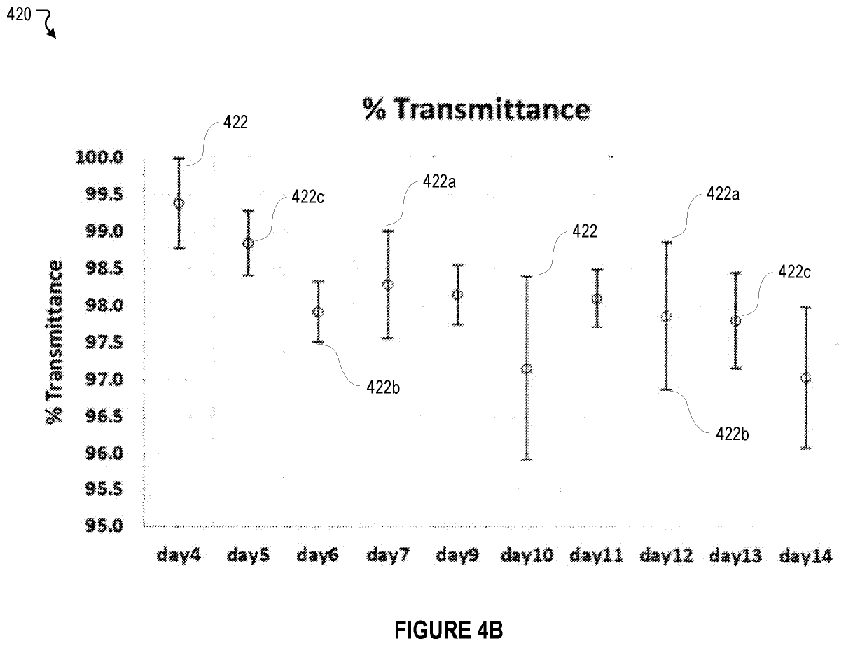

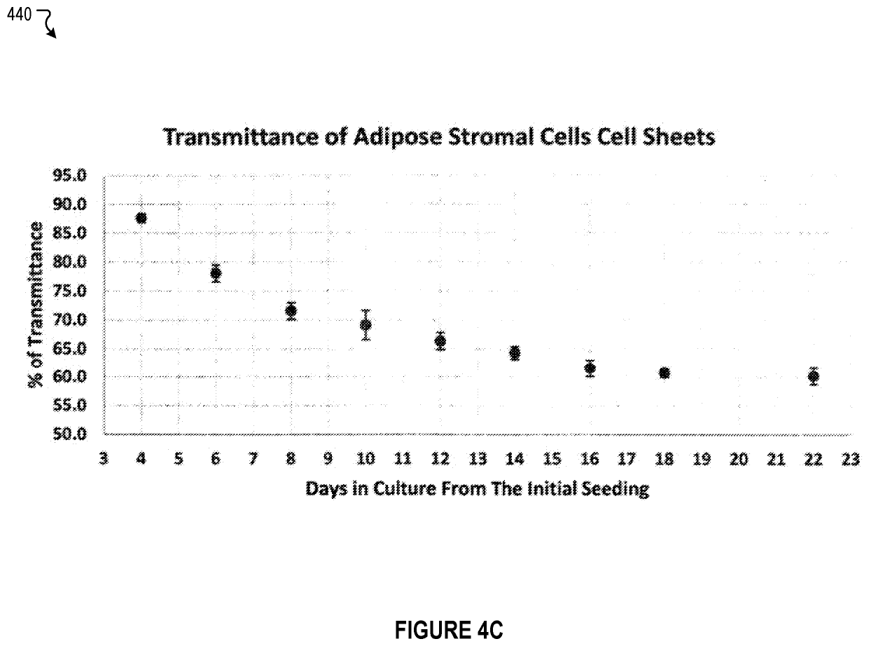

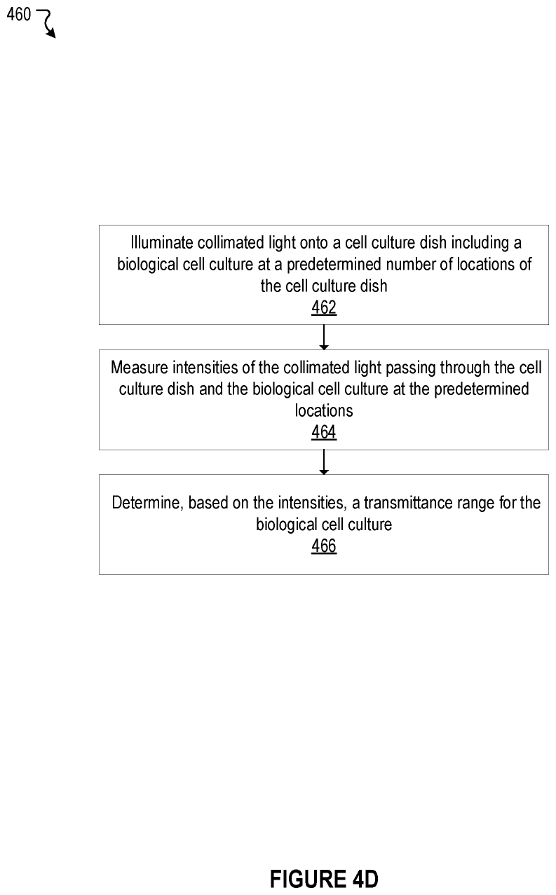

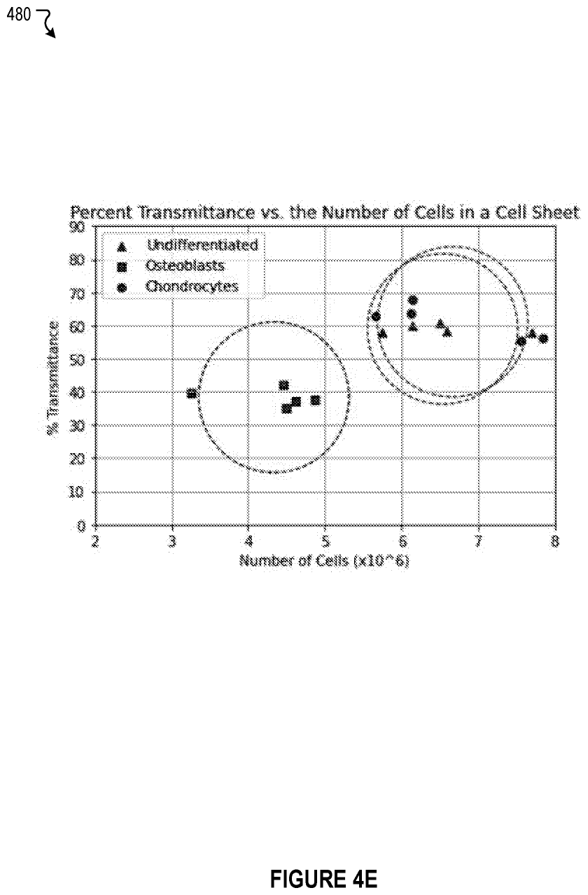

In some embodiments, the apparatus 100 can be used to measure optical density of a cornea or any other biological cell culture before transplantation. In such embodiments, a number of cells in a cell sheet can be estimated based on transmittance of the cell sheet measured using the apparatus 100 and comparing the measured transmittance with a reference graph plotting transmittance with a number of cells. In this way, when the cell sheet is harvested, quality control and release control of the cell sheet, as well as the cell sheet therapy posology, can be tightly controlled. This results in less variability of the cell sheet's maturity before harvesting. The reference graph plotting transmittance with a number of cells is further discussed in further detail with reference to FIG. 4E herein.

FIG. 4A illustrates a diagram 400 showing a relationship between transmittance (light intensity) and thickness and/or maturity of a biological cell culture, according to various embodiments of the present disclosure. In general, transmittance of light through a substance or a material is directly related to intensity of the light exiting the substance or the material. For example, a material is said to have a high transmittance of light when the light exiting the material has high light intensity. Similarly, a material is said to have a low transmittance of light when the light exiting the material has low light intensity. Furthermore, in general, thickness and/or maturity of a biological cell culture is directly related to a number of cell sheets. For example, the thicker a biological cell culture is, the more cell sheets the biological cell culture has. FIG. 4A shows a simplified diagram of the apparatus 100 in which a cell culture dish is secured onto the linear stage 108. Further, FIG. 4A shows three scenarios 402-406 of measuring light intensity. In the scenario 402, the cell culture dish including a biological cell culture has been grown in the cell culture dish for a first number of days. The percentage of transmittance used in the FIG. 4A are used as an example to describe the changes in transmittance, when the cell sheets are grown on the cell culture dish. Light generated from the light source 104 to pass through the cell culture dish and the biological cell culture loses approximately 1% of light intensity. In this scenario, the cell culture dish and the biological cell culture are said to have a transmittance of 99%. This transmittance can be correlated to thickness and/or maturity of the biological cell culture grown during the first number of days. In the scenario 404, the biological cell culture has been further grown in the cell culture dish for a second number of days after the first number of days. When the cell culture dish and the biological cell culture is placed into the apparatus 100 again to measure light intensity, this time, light exiting the cell culture dish and the biological cell culture loses approximately 3% of light intensity and the cell culture dish and the biological cell culture are said to have a transmittance of 97%. In the scenario 406, the biological cell culture has been further grown in the cell culture dish for a third number of days after the second number of days. When the cell culture dish and the biological cell culture is placed into the apparatus 100 again to measure light intensity, this time, light exiting the cell culture dish and the biological cell culture loses approximately 12% of light intensity and the cell culture dish and the biological cell culture are said to have a transmittance of 88%. As such, using this method, transmittance of a biological cell culture can be correlated to thickness and/or maturity of the biological cell culture (i.e., a number of cell sheets the biological cell culture has). Furthermore, in some cases, transmittance of a biological cell culture can be correlated to maturity of the biological cell culture. For example, the more mature a biological cell culture is (i.e., the longer the biological cell culture has been grown in a cell culture dish), the thicker the biological cell culture, and thus, better able to withstand stress associated with being removed from a cell culture dish. Based on this correlation, transmittance of a biological cell culture can be calibrated to determine thickness and/or maturity of the biological cell culture and a time for harvesting the biological cell culture with a non-invasive methodology. For example, a time for harvesting a biological cell culture can be determined based on thickness and/or maturity of the biological cell culture. In this example, the thickness and/or maturity of the biological cell culture can be determined by determining transmittance of the biological cell culture. So, in this example, the time for harvesting the biological cell culture can be determined based on the transmittance of the biological cell culture. In this way, harvesting time for biological cell cultures can be determined in a non-invasive way and without damaging the biological cell cultures.

FIGS. 4B-4C illustrate graphs 420 and 440 depicting a relationship between transmittance of a biological cell culture, such as a multilayered stem or cornea cell sheet, and days the biological cell culture was grown in a laboratory environment, according to various embodiments of the present disclosure. The graph 420 is an x-y graph. The x-axis of the x-y graph represents days the biological cell culture was grown, and the y-axis of the x-y graph represents transmittance of the biological cell culture measured at particular days. In the graph 420, variances of transmittance values measured at various points of the biological cell culture at the particular days are represented by bars 422. Maximum and minimum transmittance values are indicated by edges 422a, 422b of the bars 422. Average transmittance values are represented by circles 422c of the bars 422. Based on the average transmittance values, a mathematical relationship between transmittance and days of growth can be determined. As such, just by measuring a transmittance value of a biological cell culture, number of days that the biological cell culture was grown, and therefore thickness and/or maturity can be determined. The graph 420 is an x-y graph showing transmittance of adipose stromal cell sheet and days the adipose stromal cell sheet was grown in a cell culture dish.

FIG. 4D illustrates a method 460 of determining thickness and/or maturity of a biological cell culture, such as a multilayered stem or cornea cell sheet, according to various embodiments of the present disclosure. In step 462, collimated light is illuminated onto a cell culture dish including a biological cell culture at a predetermined number of locations of the cell culture dish. The collimated light is generated by the light source 104 of the apparatus 100 and collimated through the collimator 106 of the apparatus 100. The predetermined number of locations on the cell culture dish is determined based on a configuration setting to be loaded to the controller 120 of the apparatus 100. The cell culture dish is actuated to the predetermined number of locations using the linear stage 108 of the apparatus 100.

In step 464, intensities of the collimated light passing through the biological cell culture are measured at the predetermined number of locations. The intensities are measured by the photodetector 110 of the apparatus 100. The photodetector measures at least 10 intensity values at each of the predetermined number of locations.

In step 466, a transmittance range for the biological cell culture is determined based on the intensities. The transmittance range includes at least a maximum transmittance value, a minimum transmittance value, and an average transmittance value for the biological cell culture. Transmittance ranges for the biological cell culture measured at particular days are plotted on a graph. Based on average transmittance values measured at the particular days, a correlation curve can be determined between transmittance values of the biological cell culture and thickness/maturity and the number of cells per cell sheets, of the biological cell culture.

FIG. 4E illustrates a reference graph 480 that can be used to determine a number of cells in a cell sheet, according to various embodiments of the present disclosure. As discussed above, a number of cells in a cell sheet can be estimated based on transmittance of the cell sheet measured using the apparatus 100 and comparing the measured transmittance with the reference graph 480. As shown in FIG. 4E, the reference graph 480 can be an x-y scatter plot. The x-y scatter plot can include percent transmittance of the cell sheet on the y-axis and a number of cells in the cell sheet on the x-axis. plotting transmittance with a number of cells. Further, the x-y scatter plot can include clusters of data points from different culture media. For example, undifferentiated culture media are represented as triangular data points, osteoblasts are represented as square data points, and chondrocytes are presented as circular data points. As such, by knowing transmittance of a cell sheet and culture media that was used to grow the cell sheet, a number of cells in the cell sheet can be determined based on the reference graph 480. In this way, when the cell sheet is harvested, quality control and release control of the cell sheet, as well as the cell sheet therapy posology, can be tightly controlled.

What we claim is:

1. An apparatus for evaluating biological cell cultures, the apparatus comprising:
a housing, wherein the housing comprises:
a light source to generate light, wherein the light source is disposed at top of the housing;
a photodetector, wherein the photodetector is disposed at a base on the housing;
a collimator to collimate the light, wherein the collimator is disposed below the light source and the photodetector is to receive the collimated light; and
a linear stage to actuate a cell culture dish including a biological cell culture in orthogonal directions, wherein the linear stage is disposed between the light source and the photodetector, and provides a surface to secure the cell culture dish; and
a controller coupled to the housing over a data bus, wherein the controller is configured to provide instructions to operate the light source, the linear stage, and the photodetector over the data bus.

2. The apparatus of claim 1, wherein the housing further comprises:
rods extending vertically from the base of the housing;
a first bridge mechanically coupled to the rods, wherein the first bridge is disposed at the top of the housing and includes the light source; and
a second bridge mechanically coupled to the rods, wherein the second bridge is disposed between the first bridge and the collimator and includes an aperture aligned to a line of sight of the light source.

3. The apparatus of claim 2, wherein the collimator comprises at least one lens, and wherein the lens is mechanically coupled to a rod via an arm and an arm joint.

4. The apparatus of claim 3, wherein the lens has a lens diameter of 25.4 mm and a focal length of 25.4 mm.

5. The apparatus of claim 3, wherein the lens housed in a bracket coupled to the arm.

6. The apparatus of claim 1, wherein the linear stage comprises a sample stage, an x-stage, and a y-stage, wherein the sample stage provides the surface to secure the cell culture dish, wherein the x-stage actuates the linear stage in a first direction, and wherein the y-stage actuates the linear stage in a second direction orthogonal to the first direction.

7. The apparatus of claim 6, wherein the x-stage and the y-stage include a stepper motor coupled to a timing belt that causes the x-stage and the y-stage to move in their respective directions.

8. The apparatus of claim 6, wherein each of the sample stage, the x-stage, and the y-stage include an opening that allows the collimated light to pass through.

9. The apparatus of claim 6, wherein the controller comprises a computing unit coupled to at least two motor drive modules.

10. The apparatus of claim 9, wherein the at least two motor drive modules generate signals to actuate the stepper motors of the x-stage and the y-stage.

11. The apparatus of claim 9, wherein the computing unit receives analog signals from the photodetector over the data bus and digitizes the analog signals.

12. The apparatus of claim 9, wherein the computing unit generates digital signals to turn the photodetector on or off.

13. The apparatus of claim 1, wherein the light generated by the light source comprises a sharp peak at 450-475 nm and a flatten peak at 560-60 nm.

14. The apparatus of claim 1, wherein the data bus is a wired data connection.

15. The apparatus of claim 14, wherein the wired data connection is at least one of an ethernet, serial, or general purpose interface bus based data connection.

16. The apparatus of claim 1, wherein the data bus is a wireless data connection.

17. The apparatus of claim 14, wherein the wireless data connection is at least one of a cellular, Wi-Fi, Bluetooth, or near-field communication based data connection.

18. A method for operating the apparatus of claim 1, the method comprising:
actuating, by the controller, the linear stage to a first location corresponding to a first predetermined location of the cell culture dish;
generating, by the light source through the collimator, collimated light to pass through the cell culture dish and the biological cell culture at the first location;
receiving, by the photodetector, the collimated light passing through the cell culture dish and the biological cell culture; and
determining, by the controller, intensity of the collimated light at the first location.

19. The method of claim 18, the method further comprising:
actuating, by the controller, the linear stage to a second location corresponding to a second predetermined location of the cell culture dish;
generating, by the light source through the collimator, collimated light to pass through the cell culture dish and the biological cell culture at the second location;
receiving, by the photodetector, the collimated light passing through the cell culture dish and the biological cell culture; and
determining, by the controller, intensity of the collimated light at the second location.

20. The method of claim 19, wherein the intensity of the collimated light at the first location and the intensity of the collimated light at the second locations are used to determine:
thickness of the biological cell culture,
maturity of the biological cell culture for harvesting the biological cell culture,
number of cells present in one or more cell sheets of the biological cell culture; and
transparency of biological cell culture.
